(19) United States
(12) Patent Application Publication
(10) Pub. No.: US 2025/0241971 A1
(43) Pub. Date: Jul. 31, 2025

(54) EXTRACT OF BETA-GLUCAN-RICH SACCHAROMYCES CEREVISIAE YEAST CELL WALLS IN THE PREVENTION OF THE TOXIC EFFECTS OF MYCOTOXIN DEOXYNIVALENOL (DON)

(71) Applicant: LESAFFRE ET COMPAGNIE, Paris (FR)

(72) Inventors: Virginie MARQUIS, Ramonville Sainte Agne (FR); Julie SCHULTHESS, Lille (FR)

(73) Assignee: LESAFFRE ET COMPAGNIE, Paris (FR)

(21) Appl. No.: 18/855,016

(22) PCT Filed: Apr. 7, 2023
(86) PCT No.: PCT/EP2023/059321

§ 371 Date: Oct. 8, 2024

(30) Foreign Application Priority Data
Apr. 8, 2022 (FR) FR2203260

(51) Int. Cl. A61K 36/064; A61K 45/06; A61P 31/10

(52) U.S. Cl. CPC A61K 36/064; A61K 45/06; A61P 31/10

(57) ABSTRACT

Extracts of beta-glucan-rich Saccharomyces cerevisiae yeast cell walls, and the use thereof in the method of preventing mycotoxicoses caused by mycotoxin deoxynivalenol, in particular in a method of preventing liver and intestinal damage, and immunotoxic effects caused by this mycotoxin. The extracts of beta-glucan-rich Saccharomyces cerevisiae yeast cell walls include β-glucans and mannans, wherein the β-glucans are present in the form of a mixture of β-1,3-glucans and β-1,6-glucans.

### PARENT PATENT APPLICATION

The present patent application claims the priority of French Patent Application FR 2203260 filed on Apr. 8, 2022. The content of the French Patent Application is incorporated herein by reference in their entirety.

### TECHNICAL FIELD

The present invention relates to the field of health, in particular animal health. The invention relates more particularly to β-glucan-rich Saccharomyces cerevisiae yeast cell wall extracts and their use in preventing the toxic effects, in particular immunotoxic effects, of mycotoxin deoxynivalenol (DON) and in preventing hepatic and intestinal damage induced by DON mycotoxin in animals.

### PRIOR ART

Mycotoxins are toxic fungal secondary metabolites produced by various molds belonging mainly to the species Aspergillus, Penicillium or Fusarium. They are natural contaminants of cereals, but also fruits, vegetables, oleaginous nuts and seeds, and spices. The contamination considerably affects the production and marketing of cereals throughout the world and also impairs the quality of the products derived from contaminated seeds. Furthermore, the presence of mycotoxins in food can have harmful effects on human and animal health, which can range from gastrointestinal and renal disorders to an immune deficiency or cancer.

Deoxynivalenol (DON) is a mycotoxin of the trichothecene family (class B) produced by various fungal contaminants of the Fusarium genus, such as F. culmorum, F. graminearum and F. pseudograminearum. It is a mycotoxin which develops on the plant in the field and which is found, in particular, in cereal crops such as corn, wheat, barley, and oats, but also on rice, rye and sorghum. As the trichothecenes are thermostable and resistant to sterilization, they can be found in derived products such as animal feed, but also in flour, bread, pasta, etc., and even beer. In animals, ingestion of feed contaminated with DON is associated with damage to the immune system and intestinal mucosa, refusal to feed, and vomiting or diarrhea, which may result in reduced growth. In humans, DON food poisoning causes abdominal pain, dizziness, headache, throat irritation, nausea, vomiting, diarrhea, and blood loss in the stool. Recently, DON has been shown to increase the risk of inflammatory bowel disease (IBD) and exacerbate its symptoms (Payros et al., Archives of Toxicology (2020), DOI: 10.1007/s00204-020-02817-z).

To prevent the risk of contamination by mycotoxins, including deoxynivalenol, the authorities in charge of food safety (FDA, EFSA, CSAH, JECFA, etc.) have issued regulations on the maximum tolerable concentrations in foodstuffs intended for human and animal food.

As post-harvest control and mitigation of contamination of cereals by DON mycotoxin are not currently feasible, the best means of combatting DON is prevention in the field. Indeed, DON is produced by different species of Fusarium which develop in cereals under very specific temperature and humidity conditions. Stopping the growth of fungi by applying appropriate fungicides during conditions favorable to the development of Fusarium fungi helps prevent the secretion of DON and other mycotoxins. One approach to reducing animal exposure to mycotoxins is to reduce their bioavailability through the inclusion of detoxifying agents in their feed. In the case of DON mycotoxin, however, there are few detoxification solutions, and those that do exist are not very effective. Biotransformation solutions have been described such as microorganisms or enzymes degrading DON into less toxic metabolite(s), but only one microorganism (Coriobacteriaceae BBSH797) is currently on the market. Regarding detoxification by adsorption, there are very few products capable of adsorbing DON.

Therefore, there is still a need in the art for new strategies for preventing the toxic effects caused by ingestion of DON mycotoxin.

### SUMMARY OF THE INVENTION

Surprisingly, the present Inventors have found that extracts of β-glucan-rich Saccharomyces cerevisiae yeast cell walls significantly improve the transepithelial resistance of the epithelial barrier of porcine intestinal cells exposed to DON mycotoxin; cause a DON-induced decrease in expression of the IL8 gene (a proinflammatory cytokine) in porcine intestinal cells in vitro, pig explants ex vivo and chicken intestinal tissue in vivo; prevent these cells from undergoing the destructuring observed in untreated cells; prevent reduction in the size of intestinal villi and changes in enterocyte morphology in porcine intestinal explants; and effectively prevent hepatic lesions and reduction in villus size induced by DON mycotoxin in chickens.

Accordingly, the object of the present invention is an extract of β-glucan-rich Saccharomyces cerevisiae yeast cell walls for use in preventing mycotoxicosis caused by mycotoxin deoxynivalenol (DON) in a subject, characterized in that the extract comprises β-glucans and mannans, wherein the β-glucans are present in the form of a mixture of β-1,3-glucans and β-1,6-glucans in a total amount greater than or equal to 50% by weight; and the mannans are present in a total amount of less than 5% by weight.

The present invention also relates to an extract of β-glucan-rich Saccharomyces cerevisiae yeast cell walls for use in preventing the immunotoxic effects of mycotoxin deoxynivalenol (DON) in a subject, characterized in that the extract comprises β-glucans and mannans, wherein the β-glucans are present in the form of a mixture of β-1,3-glucans and β-1,6-glucans in a total amount greater than or equal to 50% by weight; and the mannans are present in a total amount of less than 5% by weight.

The present invention also relates to an extract of β-glucan-rich Saccharomyces cerevisiae yeast cell walls for use in preventing hepatic and/or intestinal lesions caused by mycotoxin deoxynivalenol (DON) in a subject, characterized in that the extract comprises β-glucans and mannans, wherein the β-glucans are present in the form of a mixture of β-1,3-glucans and β-1,6-glucans in a total amount greater than or equal to 50% by weight; and the mannans are present in a total amount of less than 5% by weight.

In certain embodiments, the total amount of β-glucans present in an extract of β-glucan-rich Saccharomyces cerevisiae yeast cell walls is between 50% and 90% by weight of the extract, for example between 50% and 80%, or between 50% and 70%, or even between 50 and 60%, by weight of the extract.

In certain embodiments, the total amount of mannans present in an extract of β-glucan-rich Saccharomyces cerevisiae yeast cell walls is between 1% and 5% by weight of the extract.

In certain embodiments, the amount of mannans in the extract of β-glucan-rich Saccharomyces cerevisiae yeast cell walls is such that the β-glucan/mannan ratio is between 12 and 40 (wt/wt). In particular, the β-glucan/mannan ratio may be between 15 and 30 (wt/wt), or even more particularly between 18 and 22 (wt/wt).

In certain embodiments, the extract of β-glucan-rich Saccharomyces cerevisiae yeast cell walls is characterized by having a dry matter content greater than or equal to 94% by weight. In particular, the dry matter content may be greater than or equal to 96% by weight.

In certain embodiments, the extract of β-glucan-rich Saccharomyces cerevisiae yeast cell walls further comprises a protein content less than or equal to 10% by weight and a glycogen content less than or equal to 10% by weight.

In certain embodiments, the β-glucans and the mannans are the only active ingredients of the extract of β-glucan-rich Saccharomyces cerevisiae yeast cell walls.

In certain embodiments, the extract of β-glucan-rich Saccharomyces cerevisiae yeast cell walls is Safglucan®, which comprises β-glucans and mannans, wherein the β-glucans are present in the form of a mixture of β-1,3-glucans and β-1,6-glucans in a total amount between 52 and 60% by weight; the mannans are present in a total amount between 1 and 5% by weight, and where the Safglucan® also comprises a protein content less than or equal to 10% by weight and a glycogen content less than or equal to 10% by weight.

The present invention also relates to a composition comprising an extract of β-glucan-rich Saccharomyces cerevisiae yeast cell walls and at least one bioactive agent for use in preventing mycotoxicosis caused by mycotoxin deoxynivalenol (DON) in a subject or in preventing the immunotoxic effects of mycotoxin deoxynivalenol (DON) in a subject or in preventing hepatic and/or intestinal lesions caused by mycotoxin deoxynivalenol (DON) in a subject, characterized in that the extract is as defined above.

The present invention also relates to a pharmaceutical composition comprising an extract of β-glucan-rich Saccharomyces cerevisiae yeast cell walls and at least one physiologically acceptable excipient for use in preventing mycotoxicosis caused by mycotoxin deoxynivalenol (DON) in a subject or in preventing the immunotoxic effects of mycotoxin deoxynivalenol (DON) in a subject or in preventing hepatic and/or intestinal lesions caused by mycotoxin deoxynivalenol (DON) in a subject, characterized in that the extract is as defined above.

In certain particular embodiments, the subject to which the invention applies is an animal, in particular an animal selected from farm animals and pets.

The present invention further relates to an animal feed comprising an extract of β-glucan-rich Saccharomyces cerevisiae yeast cell walls for use in preventing mycotoxicosis caused by mycotoxin deoxynivalenol (DON) in an animal or in preventing the immunotoxic effects of mycotoxin deoxynivalenol (DON) in an animal or in preventing hepatic and/or intestinal lesions caused by mycotoxin deoxynivalenol (DON) in an animal, characterized in that the extract is as defined above or in that the extract is part of a composition as defined above.

In certain embodiments, the bioactive agent is selected from the group consisting of mycotoxin binders, mycotoxin biotransformants, vitamins, minerals, trace elements, hepatoprotectants, immunoprotectants, and combinations thereof.

A more detailed description of certain preferred embodiments of the invention is given below.

### BRIEF DESCRIPTION OF THE FIGURES

FIG. 1: Monitoring of transepithelial resistance (TEER) as a function of time (T) in hours in porcine intestinal cells (IPEC-J2 line) continuously pretreated with Safglucan®, with the addition of DON mycotoxin. The TEER variations are compared to those of cells treated with DON mycotoxin alone, Safglucan® in the presence of DON mycotoxin, or two control conditions (epithelial cells alone or cells with Safglucan® alone).

FIG. 2: Level of expression of il8 mRNA, determined by quantitative PCR, by porcine intestinal cells (IPEC-J2 line) either continuously pretreated Safglucan® or not, in the presence or absence of DON mycotoxin.

FIG. 3: Micrographs of porcine intestinal cells (IPEC-J2 line), untreated (left image), treated with DON mycotoxin alone (middle image), and treated with Safglucan® and DON mycotoxin (right image).

FIG. 4: Level of expression of il8 mRNA by porcine intestinal cells (IPEC-J2 line) pretreated with various “β-glucan products,” an algal β-glucan (ALG), Safmannan® and Safglucan® in the presence and absence of DON mycotoxin (P value*<0.05).

FIG. 5: Histological analyses of pig jejunum explants (n=7). (A) Images taken by microscope of 5 μm sections made from paraffin blocks containing untreated jejunum explants, or treated with DON mycotoxin alone, Safglucan® alone, or Safglucan® and DON mycotoxin. (B) Graphs showing histological score, enterocyte morphology, and intestinal villi height according to the treatments of pig jejunum explants.

FIG. 6: Expression level of genes linked to inflammation determined from mRNA isolated from jejunum explants of untreated pigs, or treated with DON mycotoxin alone, Safglucan® alone, or Safglucan® and DON mycotoxin (n=5-10). (P value*<0.05).

FIG. 7: Images of chicken liver treated with DON mycotoxin (on the right) and untreated (on the left).

FIG. 8: Graphs showing the percentage of liver lesions observed in chickens on days 13 (A) and 28 (B) depending on the presence or absence of DON mycotoxin and/or Safglucan® in the diet.

FIG. 9: Graph showing the height of intestinal villi in the jejunum of untreated chickens or chickens treated with DON mycotoxin alone, Safglucan® and DON mycotoxin 13 days post-treatment.

FIG. 10: Expression levels of il8 (A) and ifnγ (B) genes linked to inflammation determined from jejunum mRNA of untreated chickens, or chickens treated with DON mycotoxin alone or with Safglucan® and DON mycotoxin.

### DETAILED DESCRIPTION

As mentioned above, the present invention relates to extracts of β-glucan-rich Saccharomyces cerevisiae yeast cell walls for use in preventing toxic effects, in particular immunotoxic effects, of DON mycotoxin in a subject, and in preventing hepatic and/or intestinal lesions caused by DON mycotoxin in a subject.

I—Extracts of β-Glucan-Rich Saccharomyces cerevisiae Yeast Cell Walls

The β-glucans of an extract according to the invention are obtained from a Saccharomyces cerevisiae yeast, and more particularly from a Saccharomyces cerevisiae yeast strain. Numerous strains of Saccharomyces cerevisiae are known in the art. They are widely used in the food industry for their role in the manufacture of several foods, including breads and fermented drinks. As used herein, the term “yeast strain” refers to a relatively homogeneous population of yeast cells obtained by cultivation (or multiplication) of the starting strain. A yeast strain is obtained from a clone, a clone being a population of yeast cells obtained from a single yeast cell. The cultivation of a Saccharomyces cerevisiae yeast strain can be carried out by any suitable method. Yeast cultivation methods are known in the prior art, and the person skilled in the art knows how to optimize the cultivation conditions for each strain according to its nature. Thus, a Saccharomyces cerevisiae yeast can be obtained by multiplication of a strain in an appropriate culture medium, for example as described in the reference book “Yeast Technology”, 2^{nd} edition, 1991, G. Reed and TW Nagodawithana, published by Van Nostrand Reinhold, ISBN 0-442-31892-8.

The terms “yeast cell walls” and “yeast hulls” are used interchangeably here and refer to the insoluble fraction of yeast cells, i.e., the yeast cell wall and plasma membrane. Conventionally, the yeast cell walls are obtained by a process comprising a step of autolysis or enzymatic hydrolysis, essentially by proteases, followed by a step of separation of the soluble fraction and the insoluble fraction, with the fraction isolated insoluble corresponding to yeast cell walls. The insoluble fraction can then be dried. The process for obtaining yeast cell walls is such that it preserves the structural polysaccharides of the cell wall, i.e., β-glucans and mannans, the mannans being in the form of mannoproteins. The methods for obtaining yeast cell walls are known in the art (see for example the reference work “Yeast Technology”, 2nd edition, 1991, G. Reed and T. W. Nagodawithana, published by Van Nostrand Reinhold, New York, ISBN 0-442-31892-8).

An extract of β-glucan-rich Saccharomyces cerevisiae yeast cell walls according to the invention refers to a fraction which has been extracted (or isolated) from the cell walls and which mainly contains β-glucans. β-glucans originating from the yeast cell wall are called “wall β-glucans.” They are essentially glucose polymers whose main chain glucose units are linked by β-1,3 linkages and whose branches are linked by β-1,6 linkages. Yeast β-glucans are insoluble and have low viscosity. The person skilled in the art knows how to extract β-glucans from the yeast cell wall. A common method includes successive hot extractions with a base and with an acid (such as acetic acid), followed by aqueous washes to remove any soluble compounds from the cell walls, and recovery of the insoluble material consisting of cell wall β-glucans.

An extract of β-glucan-rich Saccharomyces cerevisiae yeast cell walls according to the present invention comprises a total amount greater than or equal to 50% by weight of β-glucans in the form of a mixture of β-1,3-glucans and β-1,6-glucans. As used herein, the term “total amount greater than or equal to 50% by weight of β-glucans” means that the β-glucans represent at least half of the weight of the extract according to the invention. Thus, the total amount of β-glucans present in an extract according to the invention can represent between 50% and 90% of the weight of the extract, for example between 50% and 80%, or between 50% and 70%, or even between 50 and 60% of the weight of the extract. In certain particular embodiments, the total amount of β-glucans present in an extract according to the invention represents between 50% and 60%, for example, approximately 51%, approximately 52%, approximately 53%, approximately 54%, approximately 55%, approximately 56%, approximately 57%, approximately 58%, or approximately 59% of the weight of the extract.

An extract of β-glucan-rich Saccharomyces cerevisiae yeast cell walls according to the present invention also contains mannans in a total amount of less than 5% by weight. Mannans from the yeast cell wall are called “wall mannans”. These are yeast polysaccharides consisting mainly of mannose, and more precisely copolymers of neutral or acidic oses (with 5 or 6 carbon atoms), linked together by glycosidic bonds and linked to proteins. In the cell wall mannans of Saccharomyces cerevisiae, mannose is present in the form of a backbone of mannose residues (50 or more), α-(1,6)-linked, branched by short chains of mannose with α-(1,2) and α-(1,3)-linkages. The person skilled in the art knows how to extract mannans from the yeast cell wall. A common method includes enzymatic digestion of yeast walls with a preparation of β-glucanases (e.g., the industrial preparation GLUCANEX™), followed by separation of the hydrolysate by centrifugation and purification by ultrafiltration. Another method is based on hot chemical extraction.

As used here, the term “total amount less than 5% by weight of mannans” means that the mannans represent at most 5% of the weight of the extract according to the invention. Thus, the total amount of mannans present in an extract according to the invention can represent between 0.5% and 5% of the weight of the extract. In certain particular embodiments, the total amount of mannans present in an extract according to the invention represents between 1% and 5%, for example, approximately 1%, approximately 2%, approximately 3%, approximately 4%, or approximately 5% of the weight of the extract.

Preferably, an extract of β-glucan-rich Saccharomyces cerevisiae yeast cell walls according to the present invention comprises β-glucans and mannans, wherein the β-glucans are present in the form of a mixture of β-1,3-glucans and β-1,6-glucans in a total amount greater than or equal to 50% by weight; and the mannans are present in a total amount such that the β-glucan/mannan ratio is between 12 and 40 (wt/wt). In particular, the β-glucan/mannan ratio in an extract according to the invention may be between 15 and 30 (wt/wt), and more particularly between 18 and 22 (wt/wt).

In certain embodiments, an extract of β-glucan-rich Saccharomyces cerevisiae yeast cell walls according to the present invention further comprises a protein content less than or equal to 10% by weight and a glycogen content less than or equal to 10% by weight.

In certain embodiments, an extract of β-glucan-rich Saccharomyces cerevisiae yeast cell walls according to the present invention comprises, as the sole active ingredients, β-glucans and mannans, wherein the β-glucans are present in the form of a mixture of β-1,3-glucans and β-1,6-glucans in a total amount greater than or equal to 50% by weight; and the mannans are present in a total amount of less than 5% by weight such that the β-glucan/mannan ratio is between 12 and 40 (wt/wt), in particular between 15 and 30 (wt/wt), and more particularly between 18 and 22 (wt/wt).

In certain particular embodiments, an extract of Saccharomyces cerevisiae yeast cell walls according to the invention has a dry matter content greater than or equal to 94% by weight, preferably greater than or equal to 96% by weight. A dry matter content greater than or equal to 94%, preferably 96% by weight, allows better conservation of the yeast hulls, in particular better bacteriological stability and better stability with respect to undesirable reactions, whether of enzymatic origin or not.

In certain particular embodiments, an extract of Saccharomyces cerevisiae yeast cell walls according to the invention is in the form of a powder.

In certain particular embodiments, the extract of β-glucan-rich Saccharomyces cerevisiae yeast cell walls according to the invention is Safglucan® produced by the Applicant, Lesaffre. Safglucan® comprises β-glucans and mannans, as the sole active ingredients, wherein the β-glucans are present in the form of a mixture of β-1,3-glucans and β-1,6-glucans in a total amount between 52 and 60% by weight; and the mannans are present in a total amount between 1 and 5% by weight. Safglucan® also comprises a protein content less than or equal to 10% by weight and a glycogen content less than or equal to 10% by weight.

After manufacture, an extract of Saccharomyces cerevisiae yeast cell walls according to the invention may be packaged and/or stored under suitable conditions, generally in a dry and cool place, before use. The shelf life of a cell wall extract according to the invention, in suitable packaging, is 2 years from the date of production.

II—Compositions Comprising an Extract of β-Glucan-Rich Saccharomyces cerevisiae Yeast Cell Walls

In certain embodiments, the extracts of β-glucan-rich Saccharomyces cerevisiae yeast cell walls described herein are used as such. In other embodiments, the extracts of β-glucan-rich Saccharomyces cerevisiae yeast cell walls described herein are used in combination with at least one other bioactive agent. Consequently, the present invention relates to a composition comprising an extract of β-glucan-rich Saccharomyces cerevisiae yeast cell walls, as described herein, combined with at least one bioactive agent.

The bioactive agent may, for example, be chosen from agents having an effect on mycotoxins other than DON, as animal feed may be contaminated with more than one mycotoxin. Alternatively, the bioactive agent may be known to have a beneficial action on animal health. Thus, the bioactive agent can be a mycotoxin detoxification agent, an antioxidant, a hepatoprotectant, an immunoprotectant, or a combination of these agents.

As used herein, the term “mycotoxins other than DON” refers to toxins produced by various species of microscopic fungi, such as molds (Aspergillus sp., Fusarium sp. Stachybotrys sp., Penicillium sp., etc.), wherein the toxins are not deoxynivalenol. Indeed, contaminated foods often contain a mixture of mycotoxins because (1) a fungus can produce several different mycotoxins at the same time (Fusarium fungi, which secrete DON, are for example known to co-produce other mycotoxins, notably zearalenone), (2) a food can be contaminated by several fungi (cereals are often affected by Aspergillus spp.), and (3) each of the raw materials used in food can host at least one fungus producing at least a mycotoxin. Mycotoxins can develop on many substrates: fodder, cereals, oilseeds, protein crops, silage, secondarily molasses and mineral vitamin feed. The main mycotoxins are aflatoxins, ochratoxin A, patulin, citrinin, Fusarium toxins (such as fumonisins, zearalenone, trichothecenes including T2 and HT2 toxins), Alternaria toxins, ergot alkaloids, sterigmatocystin, paxillin, enniatin and beauvericin.

The mycotoxin detoxification agent present in a composition according to the invention can be selected from mycotoxin binders, mycotoxin biotransformants, and combinations thereof.

The terms “mycotoxin binding agent” and “mycotoxin binder” are used interchangeably herein. They refer to an agent that adsorbs and/or deactivates mycotoxins other than DON, for example mycotoxins present in an animal feed or animal feed ingredient, thereby reversing the harmful effects of the mycotoxins. Mycotoxin binders reduce exposure to mycotoxins by decreasing their bioavailability, resulting in reduced absorption of mycotoxins by the animal, and thus reduced distribution into the blood and target organs. The term “bioavailability,” as used here in reference to a mycotoxin, refers to the fraction of mycotoxin that is absorbed/absorbable or assimilated/assimilable by an animal. The adsorption of mycotoxins by a binding agent decreases the bioavailability of the mycotoxins because the binder-mycotoxin complex passes through the digestive system of the animal and is excreted by the animal without there having been assimilation.

Examples of mycotoxin binders include, but are not limited to, mycotoxin adsorbents selected from the group consisting of aluminosilicates (e.g., kaolinite, hydrated calcium/potassium/sodium aluminosilicate, hydrated sodium calcium aluminosilicate (HSCAS)), bentonites (e.g., sodium bentonite, calcium bentonite), montmorillonites (e.g., sodium and calcium montmorillonite), zeolites (e.g., clinoptilolite zeolite), activated carbons, micronized fibers and polymers (e.g., cholestyramine, polyvinylpyrrolidone), activated diatomaceous earth, plant fibers (e.g., wheat and alfalfa bran fibers), polysaccharides (e.g., glucomannan or its esterified forms), certain algae, certain bacteria (lactic acid bacteria) and combinations thereof.

The term “mycotoxin biotransformer” refers to an agent (usually an enzyme, bacteria, fungus), which deactivates or inactivates mycotoxins other than DON, for example mycotoxins present in animal feed or animal feed ingredients, thus reversing the harmful effects of mycotoxins.

Examples of mycotoxin biotransformants include enzymes that degrade mycotoxins other than DON, for example selected from the group consisting of esterases, lipases, proteases, oxidases, cellulases, epoxidases, dehydrogenases, hemicellulases (or xylases), catalases, peroxidases, laccases, xylanases, carboxylesterases, amino-/acetyltransferases, pancreatin, lactonases, lactonohydrolase and combinations thereof.

Other examples of mycotoxin biotransformants include microorganisms which are known to be deleterious to mycotoxins, for example selected from: Eubacterium sp. BBSH 797 (a Coriobacteriaceae), Nocardia asteroides, Mycobacterium fluoranthenivorans sp., Rhodococcus erythropolis, bacilli of the genus Alcaligenes, Bacillus, Lactobacillus, Achromobacter thermophilus C5 and NG40Z, Lactobacillus paraplantarum, Stenotrophomonas maltophila, Saccharomyces cerevisiae, Exophiala spinifera, Cupriavidus basilensis OR16 Aspergillus niger, Eurotium herbariorum, Rhizopus, Trichosporon mycotoxinivorans, Phaffia rhodozymyllces, Phaffia rhodozymyllces, Phaffia rhodozymhousces, Nocardia corynebacterioides NRRL 24037, Mycobacterium fluoranthenivorans or Myxococcus fulvus ANSM068, Rhodococcus erythropolis, Brevibacterium sp., Slackia sp. DG6, Deviosa mutans, Deviosa insulae A16, Solanum tuberosum, Aspergillus oryzae, Eggerthella sp. DII-9, Pseudomonas sp. Y1, Lysobacter sp. S1, Sphingomonas, Nocardioides, Citrobacter, Marmaricola sp. MIM116 and combinations thereof.

In certain embodiments, the bioactive agent present in a composition according to the present invention is an antioxidant known to reduce the toxicity of mycotoxins other than DON in animals. Examples of such bioactive agents include, but are not limited to, rutin, quercetin, lutein, lecithin, melatonin, mannitol, curcumin, curcumoids, lycopene, allyl sulfides, fructose, chlorophyll and its derivatives, sodium thiosulfate, glutathione, methionine, aspartame, trace elements (selenium, zinc, magnesium), catechin (epigallocatechin gallate, epicatechin gallate), morin, kaempferol, fisetin, naringin, vitamins (vitamins E, C, A and B), coenzyme Q10, provitamins (carotene and carotenoids), eugenol, vanillin, caffeic acid, cholinergic acid, and combinations thereof.

In certain embodiments, the bioactive agent is a hepatoprotectant or immunoprotectant known to be active in animals. The terms “hepatoprotectant” and “liver protectant” are used interchangeably here. They refer to an agent which improves the integrity and regeneration of hepatocytes, optimizing the detoxification capacity of the liver and/or which promotes hepatic synthesis by stimulating the activity of digestive enzymes which ensure optimal use of nutrients in increasing their intestinal absorption and, therefore, their bioavailability. Examples of hepatoprotectants include, but are not limited to, liver protectants of natural origin composed of a combination of a variable number of plants having different hepatoprotective properties such as Phyllanthus niruri, Azadirachta indica, Andrographis paniculata, Achyrantes aspera, etc., and liver protectants that donate methyl groups based on the ability of methyl groups to bind to toxins, thus promoting their elimination from the body. Among the compounds capable of donating methyl groups, we distinguish certain amino acids and their derivatives (for example, methionine, carnitine, betaine, etc.), vitamin derivatives (for example, choline). The term “immunoprotectant” refers to an agent which, whatever its mechanism of action, protects against the effects of an antigen. Examples of immunoprotective agents include, but are not limited to, envelope proteins derived from animal viruses; oligonucleotides such as for example CpG oligonucleotides. Immunoprotectants may also be chemical immunoprotectants which include, but are not limited to, cytokines, chemokines, and lymphokines, including, but not limited to, interferon alpha, interferon gamma, and interleukin 12, or immunoprotectants of plant origin, such as plants: Eclipta alba, Aloe vera, Ocimum sanctum, Viscum album, Urtica dioica and Zingiber officinale, Solanum trilobatum, Astragalus radix and Scutellaria radix, and Achyranthes aspera.

In a composition according to the invention, the bioactive agent(s) are generally present in an amount which is sufficient to achieve the desired goal (for example reduction of the bioavailability of mycotoxins and/or the inactivation of mycotoxins). A person skilled in the art knows how to determine such an amount. For example, the enzyme(s) which degrade mycotoxins other than DON may be present in an amount less than or equal to 5% by weight of enzyme relative to the total weight of the composition, preferably less than 1%, more preferably between 0.01% and 0.5%, more preferably between 0.15% and 0.25% by weight of enzyme relative to the total weight of the composition. One or more bioactive agents binding mycotoxins other than DON may, for example, be present in an amount of up to approximately 90-95% by weight relative to the total weight of the composition, for example approximately 80%, approximately in total weight relative to the total weight of the composition, for example approximately 70%, approximately 60%, approximately 50%, approximately 45%, approximately 40%, approximately 35%, approximately 30%, approximately 25%, approximately 20%, approximately 15%, approximately 10%, or less than 10% by weight relative to the total weight of the composition.

In certain embodiments, a composition according to the invention may also comprise components which are generally found in supplements, complements or feed additives for animals, such as for example vitamins, minerals, trace elements, etc. The person skilled in the art knows how to select the appropriate components depending on the animal for which the composition is intended and knows how to determine the appropriate quantities to be included in such a composition.

The compositions according to the invention can be in any form, for example in the form of powder, granules, a gel or a liquid. Preferably, the compositions according to the invention are in solid form, preferably in powder form.

The invention also relates to a pharmaceutical composition comprising an extract of β-glucan-rich Saccharomyces cerevisiae yeast cell walls, as described herein, and at least one physiologically acceptable excipient in veterinary medicine. In the pharmaceutical composition, the extract may be present as the only active ingredient. Alternatively, the pharmaceutical composition may also comprise at least one other bioactive agent, such as those listed above.

A pharmaceutical composition according to the invention can be classified as a veterinary pharmaceutical preparation available by prescription or over the counter.

In the context of the present invention, the term “physiologically acceptable excipient in veterinary medicine” refers to any medium or additive which does not interfere with the effectiveness of the biological activity of the active principle (here the β-glucan-rich yeast cell wall extract) and which is not excessively toxic to the animal at the administered concentrations.

The veterinary pharmaceutical compositions according to the present invention can be administered using any combination of dosage and administration route that is effective in achieving the desired prophylactic effect. The person skilled in the art will recognize that the exact amount to be administered may vary from one animal species to another and will know the effects of DON mycotoxin on the animal species to be treated.

III—Uses of Extracts of β-Glucan-Rich Saccharomyces cerevisiae Yeast Cell Walls

As noted above, DON has a negative impact on the intestine and immunological responses. This mycotoxin induces intestinal histological damage, including necrosis of the intestinal epithelium. DON also disrupts intestinal barrier function, which may lead to increased translocation of pathogens and greater susceptibility to enteric infectious diseases. DON also modulates the immune reactivity of the intestinal mucosa, can interact in the dialogue between epithelial cells and intestinal immune cells and represents a predisposing factor to inflammatory diseases.

It has been shown that the β-glucan-rich Saccharomyces cerevisiae yeast cell wall extracts described herein, which do not adsorb DON mycotoxin, are able to significantly improve the transepithelial resistance of the epithelial barrier of porcine intestinal cells exposed to DON mycotoxin; to cause a decrease in the expression of the il8 gene (a pro-inflammatory cytokine) induced by DON in porcine intestinal cells in vitro, in pig explants ex vivo and in chicken intestinal tissue in vivo; to prevent these cells from undergoing the destructuring observed in untreated cells; to prevent reduction in the size of intestinal villi and damage to enterocyte morphology in porcine intestinal explants; and to effectively prevent liver damage and reduction in villus size induced by DON mycotoxin in chickens.

The invention therefore also relates to an extract of β-glucan-rich Saccharomyces cerevisiae yeast cell walls, as described herein, alone or in combination with at least one bioactive agent, for use in preventing mycotoxicosis caused by DON mycotoxin in a subject. The invention also relates to an extract of β-glucan-rich Saccharomyces cerevisiae yeast cell walls as described herein, alone or in combination with at least one bioactive agent, for use in preventing the toxic effects, in particular immunotoxic effects, of DON mycotoxin in a subject. The invention also relates to an extract of β-glucan-rich Saccharomyces cerevisiae yeast cell walls as described herein, alone or in combination with at least one bioactive agent, for use in preventing liver and/or intestinal lesions induced by DON mycotoxin in a subject.

The term “mycotoxicosis” refers to a condition occurring due to the ingestion of a mycotoxin which comes from food contaminated with said mycotoxin produced by a microscopic fungus. Mycotoxicosis caused by DON mycotoxin is therefore a condition occurring due to the ingestion of DON mycotoxin. As used herein, the term “preventing mycotoxicosis caused by DON mycotoxin in a subject” refers to reducing or inhibiting, partially or entirely, the likelihood that a subject will develop one or more symptoms related to the ingestion of DON (reduction in growth, immunosuppression, vomiting, diarrhea, damage to the intestinal mucosa, refusal to feed, liver damage, abdominal pain, etc. depending on the subject).

The term “immunotoxic effects of DON mycotoxin”, as used herein, refers to the deleterious effects induced by the ingestion of DON on the immune system. Different types of immunotoxic effects are possible, including immunosuppression which can promote infections and tumors, immunostimulation, hypersensitivity and autoimmunity. When animals are exposed to low doses of DON, certain components of the immune system are stimulated. Conversely, when animals are exposed to higher doses of DON, an immunosuppressive reaction is generally observed. The term “preventing the immunotoxic effects of DON mycotoxin” refers to reducing or inhibiting, partially or entirely, the probability that a subject will experience an immunotoxic effect after ingestion of DON.

The term “hepatic and/or intestinal lesions,” as used herein, refers to any pathological modification of a tissue or cell of the liver and/or intestines, which are then in an abnormal state. This damage can cause liver and/or intestinal dysfunction.

As used herein, the term “subject” refers to a human or an animal. As used herein, the term “animal” refers to a living being of the kingdom Animalia. In the context of the present invention, the term refers more precisely to livestock (such as cattle (cow, buffalo, zebu, bison, aurochs, yak), ovines (sheep), caprines (goat), porcines (pig), equines (horse, donkey, mule), camelids (camel, dromedary, llama, alpaca), and cervids (reindeer, deer)) and other livestock (chicken, turkey, duck, goose, pigeon, quail, pheasant, partridge, ostrich, emu, rhea, ratite, guinea fowl, rabbit, guinea pig); aquatic animals (marine, pond or freshwater fish farming, shellfish farming, crustacean farming and mollusk farming); pets (cats, dogs, rabbits, horses, etc.); and laboratory animals. The term animal, as used herein, does not refer to a particular age.

In certain particular embodiments, the animal for which the Saccharomyces cerevisiae yeast cell wall extracts described here are intended is chosen from pets and farm animals, in particular livestock (pig, cattle, sheep, goats) and poultry (chicken, turkey, rooster, guinea fowl, quail, duck, goose).

Animals do not all have the same sensitivity to DON mycotoxin. Differences in sensitivity are notably due to differences in the absorption mechanism, metabolism and distribution and elimination of DON. Pigs are known to be very sensitive to DON due to their diet rich in cereals and also because the mycotoxin is quickly and efficiently absorbed before being distributed to the organs while being only weakly metabolized and therefore detoxified. Poultry are less sensitive than pigs to DON. The first zootechnical effects are observed from a concentration of 5 mg of DON per kilogram of feed. However, acute poisoning results in the death of the animal between 3.5 hours and 13.5 hours after ingestion of the contaminated feed. For ruminants, contamination by DON is possible through cereal concentrates or through corn silage containing DON. Other fodder (hay, haylage) may also be contaminated, but the development of Fusarium and the production of DON after harvest are considered negligible. Ruminants appear to have little sensitivity to DON up to 12 mg/kg of feed.

In certain embodiments of the present invention, the use of an extract of β-glucan-rich Saccharomyces cerevisiae yeast cell walls, alone or combined with at least one bioactive agent, in preventing mycotoxicosis caused by DON mycotoxin in a subject and/or in preventing immunotoxic effects of DON mycotoxin in a subject and/or in preventing liver and/or intestinal lesions induced by DON mycotoxin in a subject comprises mixing a β-glucan-rich Saccharomyces cerevisiae yeast cell wall extract with an animal feed. Mixing can be carried out by any suitable method known in the art.

As used herein, the terms “animal feed,” “animal feed ingredient” and “animal feed product” refer to any natural or processed organic material that is susceptible to biodegradation and can be consumed by an animal. Examples of such organic materials range from freshly harvested grains to pelleted feeds, and include for example any compound, grain, nut, forage, silage, preparation, composition or mixture that is suitable or intended for ingestion by an animal. The term “fodder” refers to plant material (mainly leaves and stems of plants) intended for ruminants. The term “silage” refers to fermented forage with a high moisture content that can be used as feed for ruminants. The preparations, compositions and mixtures, e.g., commercial preparations, compositions and mixtures, may be in any suitable form, e.g., in the form of concentrates, premixes, supplements, total mixed ration (TMR), etc. The animal feed may contain cereals (e.g., corn, wheat, barley, rye, rice, sorghum, millet or any combination thereof), fodder, silage, legumes (e.g., soya), spent grains, animal products, etc. Spent grains mainly come from industrial processes such as brewing and distilleries intended for the production of drinkable alcohol (whiskey, vodka or gin) or biofuels. Brewers' grains are produced from malt, itself derived from barley. Cereals other than barley, such as sorghum, can occasionally be used in brewing, particularly in Africa. The spent grains from the manufacture of bioethanol are made from corn or wheat. Wet spent grains are dried to produce dry grains, which are mainly used as animal feed.

In certain embodiments, the animal feed to which a β-glucan-rich Saccharomyces cerevisiae yeast cell wall extract as described herein is added has been determined to be contaminated with molds capable of producing DON mycotoxin or containing DON mycotoxin. In other embodiments, the animal feed has been determined to be susceptible to contamination with molds capable of producing DON mycotoxin or susceptible to containing DON mycotoxin. In yet other embodiments, nothing is known regarding the potential contamination of the animal feed by molds capable of producing DON mycotoxin or by DON mycotoxin.

A β-glucan-rich Saccharomyces cerevisiae yeast cell wall extract, as described herein, alone or in combination with at least one bioactive agent, may be added to an animal feed in amounts from about 0.003% to about 0.5% by total weight of feed (which corresponds to amounts of approximately 0.03 kg to approximately 5 kg per ton of feed) subject to compliance with current legislation. For example, in some embodiments, a β-glucan-rich Saccharomyces cerevisiae yeast cell wall extract as described herein, alone or in combination with at least one bioactive agent, is added to an animal feed in an amount from approximately 0.003% to approximately 0.5% by total weight of the feed (which corresponds to an amount of approximately 0.03 kg to approximately 5 kg per ton of feed), for example in an amount of approximately 0.005% to approximately 0.05% by weight of feed (which corresponds to approximately 0.05 kg to approximately 0.5 kg per ton of feed).

In the above, emphasis has been placed on the use of β-glucan-rich Saccharomyces cerevisiae yeast cell wall extracts in the field of animal health. However, it is planned to extend the use of these extracts to human health, as deoxynivalenol is responsible for serious mycotoxicosis in humans.

Unless otherwise defined, all technical and scientific terms used in the Description have the same meaning as that commonly understood by an ordinary specialist in the field to which this invention belongs. Likewise, all publications, patent applications, patents and other references mentioned herein are incorporated by reference.

### EXAMPLES

The following examples describe certain embodiments of the present invention. However, it is understood that the examples and figures are presented for illustrative purposes only and in no way limit the scope of the invention.

In all examples below, the term “CTL” refers to “control.”

### Example 1: Evaluation of Safglucan® In Vitro

In vitro studies were carried out on the porcine intestinal cell line IPEC-J2, and the effects on membrane permeability and on the expression of different genes were studied. Due to their grain-rich diet, pigs are exposed and sensitive to DON mycotoxin to a particular degree. In addition, pigs are a good model for humans, particularly in terms of digestion and immunity.

### Materials and Methods

Various “β-glucan products” were used in this study: Safglucan®, according to the present invention, which is produced by Lesaffre, the Applicant; an algal β-glucan, and Safmannan® produced by Lesaffre. The characteristics of these “β-glucan products” are shown in Table 1 below.

TABLE 1
Characteristics of the “β-glucan products” studied.

| | | | | | | β-glucan/ |
|---|---|---|---|---|---|---|
| | | | | β-glucans | Mannans | mannan |
| Name | Description | Structure | Origin | (in %) | (in %) | ratio |
| Safglucan ® | β-1,3/1,6- | Branched | S. cerevisiae | 57.4 | 1.46 | 39 |
| | glucans | | | (52-60) | (1-5) | (12-40) |
| Algal | β-1,3 | Linear | Euglena | 56.1 | 3.45 | 16.2 |
| β-glucan | glucan | | gracilis | | | |
| Safmannan ® | β-1,3/1,6- | Branched | S. cerevisiae | 22.4 | 22.3 | 1 |
| | glucans | | | | | |

IPEC-J2 cells were thawed in DMEM/F12 medium+5% SVF+16 mM Hepes+1×ITS+5 ng/ml EGF and maintained in culture for 2 passages (1 week). On the third passage, the cells were cultured in the presence of pig serum (Gibco) and maintained for 2 passages (1 week).

A “β-glucan product” was added to IPEC-J2 cells at a final concentration of 50 μg/mL and this concentration was maintained at each passage. After 15 days of culture in the presence of a “β-glucan product”, various tests were carried out: a TEER (transepithelial/endothelial electrical resistance) test and mRNA extraction for quantification of IL8 by quantitative PCR.

DON mycotoxin was prepared every 2 to 3 weeks from a frozen and parafilmed stock solution (30 mM DON in acetonitrile) as follows. The DON solution was removed from the freezer 2 minutes before sampling and vortexed. It was verified that there was no crystal formation. 30 μL of the solution was removed and added to 270 μL of sterile water, yielding the stock solution at 3 mM in a water/acetonitrile mixture (10/90, v/v). The stock solution was diluted one third in a water/acetonitrile mixture (10/90, v/v) to obtain a 1 mM solution. These two solutions were then used diluted 1/100 in the wells of the TEER tests and the wells of the 96 microwell plates of the quantitative PCR tests to final concentrations of 10 and 30 μM.

TEER Trial Protocol. In this trial, Falcon 24-well inserts were used. The wells were pre-moistened by placing 800 μL of medium basolaterally, wetting the membrane with 100 μL of medium and incubating for 15 minutes at 37° C. Cell suspensions containing 0.5×10⁵ cells/mL cultured continuously with a β-glucan product were prepared, and 300 μL of each of these suspensions was placed in each pre-moistened insert. Three days later, the medium was changed in each insert. The inserts were placed in a cellZscope® and the TEER value was determined. The next day, DON mycotoxin was added, and the TEER was read for at least 24 hours.

RNA for Quantitative PCR. Cell suspensions containing 0.5×10⁵ cells/mL cultured or not with a continuous β-glucan product were prepared and 200 μL of each of these suspensions was deposited in each well of a 96 microwell plate (i.e., 10,000 cells/well). The next day, DON mycotoxin was added in the morning and incubated for 6 hours. The medium was then removed by aspiration and 130 μL of RA1+5 μL of TCEP was added to each well to lyse the cells. The plate was stored at −20° C. until the day of RNA extraction with the Nucleospin™ 96 RNA kit from Macherey Nagel™.

RNA Extraction and Reverse Transcription. RNA extraction was carried out by centrifugation following the instructions provided in the Macherey-Nagel® NucleoSpin™ 96 RNA Kit package insert (reference 740709.4). Elution was carried out in 30 μL of RNase-free water. 10 μL of RNA was then used to carry out reverse transcription into complementary DNA using the Applied Biosystems™ High-Capacity cDNA Reverse Transcription Kit (reference 4368814).

A reaction, in the absence of RNase inhibitor, contains: 2 μL of 10×RT Buffer, 0.8 μL of 25×dNTP Mix (100 mM), 2 μL of 10×RT random Primers, 1 μL of MultiscLribe Reverse Transcriptase and 4.2 μL of nuclease-free water.

The thermocycling program used was as follows: 10 minutes at 25° C., 2 hours at 37° C., 5 minutes at 85° C. and maintenance at 4° C.

Quantitative PCR. PCRs were carried out on a QuantStudio 3 device (Applied Biosystems). The master mixes (Applied Biosystems™ Taqman™ Fast Advanced Master Mix, ref. 4444964), Taqman™ primers and probes (Taqman gene expression assay, HPRT pig, Ss03388274-m1-VIC-MGB-PL; Taqman gene expression assay, IL8 pig, Ss03392437_m1-FAM-MGB) were ordered from ThermoFisher. The PCRs carried out are relative quantitative PCRs which compare the expression of the il8 gene in the presence and absence of the “β-glucan product”. The results obtained are expressed in 2^{-deltaCT}, delta Ct being the difference in Ct between the gene of interest (il8) and the housekeeping gene (HPRT) whose expression is stable.

### Results

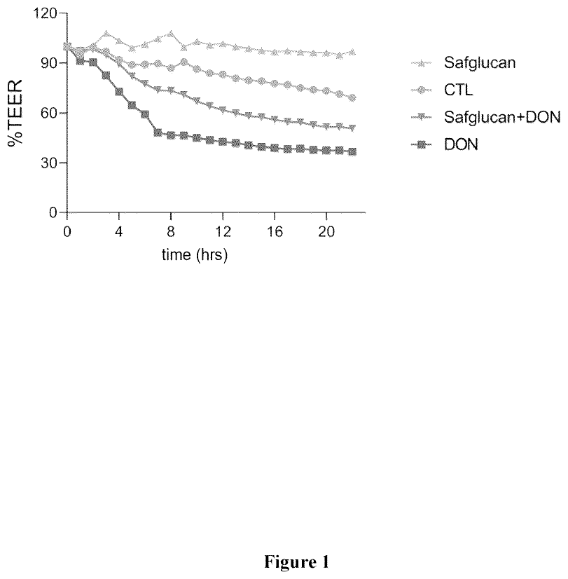

The results of the in vitro studies are shown in FIGS. 1 to 4.

The porcine intestinal cell line IPEC-J2 was continuously pretreated with Safglucan® with the addition of DON mycotoxin. The results of the TEER tests, shown in FIG. 1, express the transepithelial resistance of the epithelial barrier of IPEC-J2 and show that cells pretreated with Safglucan® are less affected by the effects of DON mycotoxin than non-pretreated cells.

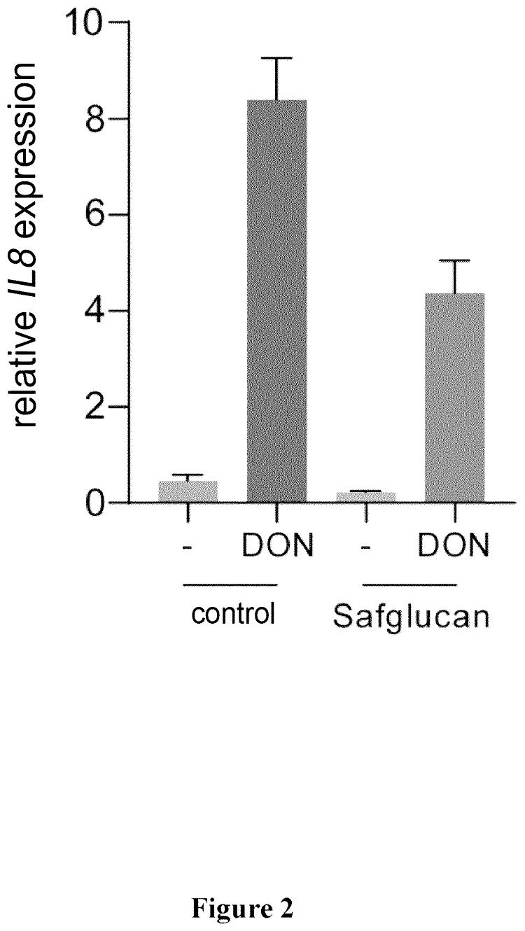

FIG. 2 shows quantitative PCR results on the expression of il8 (a pro-inflammatory gene that is expressed by epithelial cells when in danger). These results show that continuous pretreatment of IPEC-J2 cells with Safglucan® leads to a decrease in IL-8 production induced by DON mycotoxin in IPEC-J2.

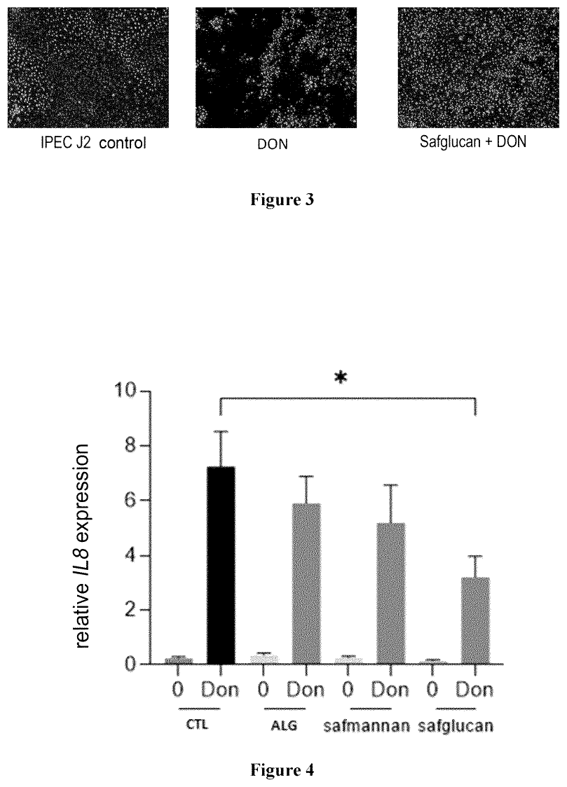

Micrographs (shown in FIG. 3) show that IPEC-J2 cells treated with Safglucan® in the presence of DON mycotoxin are less de-structured than cells treated with DON mycotoxin alone.

The porcine intestinal cell line IPEC-J2 was then pretreated continuously with Safglucan®, as well as with other “β-glucan products” having different origins and/or β-glucan/mannan ratios, before addition of DON mycotoxin. The expression of interleukin IL-8 by IPEC-J2 was measured. The results, shown in FIG. 4, indicate that cells pretreated with Safglucan® are less affected by the effects of DON than cells pretreated with other “β-glucan products”.

### Example 2: Adsorption In Vitro of Mycotoxin DON by Safglucan®

In order to understand how Safglucan® works, a test was carried out in vitro to determine the capacity of Safglucan® to adsorb DON mycotoxin. Incubation was carried out at two different pHs (3 and 7) and for two different batches of Safglucan® (batch 1 and batch 2). The results obtained are shown in Table 2 below.

TABLE 2
Adsorption of DON mycotoxin by Safglucan ®.

| | % adsorption at pH 3 | % adsorption at pH 7 |
|---|---|---|
| Safglucan ® batch 1 | 1% | 5% |
| Safglucan ® batch 2 | 0.5% | 2% |

These results make it possible to exclude a mechanism of action via adsorption of DON mycotoxin by Safglucan® whether under acidic pH conditions (pH 3) or under neutral pH conditions (pH 7). By comparison, it was determined that the adsorption of mycotoxin by the Safmannan® product is <10%.

### Example 3: Evaluation of the Effects of Safglucan® Ex Vivo

The experiments described in this example were carried out by the team of Dr Isabelle Oswald, whose address is as follows: INRAE—NATIONAL RESEARCH INSTITUTE for AGRICULTURE, FOOD and the ENVIRONMENT, Food Toxicology Unit UMR 1331 ToxAlim—180 chemin de Tournefeuille—BP93173-31027 Toulouse Cedex 03.

Er vivo studies were carried out on pig intestinal explants to determine the effects on intestinal tissue and the expression of different genes. Explant culture is an alternative method between cell culture and the animal as a whole. It makes it possible to test for contaminants/additives on a whole organ sample containing the different cell types that compose it. It also makes it possible to carry out repetitions and limit the variability of the responses observed (each animal being its own control).

### Materials and Methods

Experimental protocol. The animals were raised under feeding and environmental conditions that best represent those of farmed animals. The jejunum explants were exposed to DON mycotoxin in the presence or absence of Safglucan® at 10 mg/L⁻¹. Segments of jejunum were removed from 6 to 12 pigs aged 4 weeks and quickly placed in washing medium (cold Williams E medium+antibiotics: penicillin, streptomycin and gentamicin). The explants were made using biopsy punches 8 mm in diameter for histological analyses and 6 mm in diameter for qPCR and Western blot analyses, taking care to identify the mucosal vs. muscular orientation. The explants were then delicately placed in 6-well plates containing complete medium (Williams E medium+glucose (25 g/L), 1% ITS, 1% Ala-Glu (3 mol/L), 1% penicillin/streptomycin and 0.5% gentamicin) on sponges for 4 hours (the time could be modified depending on the analyses to be carried out) at 39° C. (the temperature could be modified depending on the analyses to be carried out) on a shaking tray.

The jejunal explants were exposed or not to DON (10 μM) and co-treated or not with 10 mg/L of Safglucan® solution. The dose of 10 μM in DON was chosen because it corresponds to a concentration of 3 mg/L (3 ppm), which is close to the concentrations that can be found in animal feed. In fact, the maximum levels of deoxynivalenol recommended in Europe in foods are 5 ppm for complete feeds, with the exception of the feed for these pigs with a dose of 0.9 ppm.

On completion of processing, the samples were collected and stored as follows:
For histological analyses: the biological samples were transferred into cassettes and the tissues were fixed in 4% formalin before transfer to a 70% alcohol solution, followed by embedding in paraffin. Measurement of the depth of the crypts and the length of the villi was also carried out using the image analysis software NIS-Elements AR (Nikon System-Elements Advanced Research).
For quantitative PCR and Western Blot analyses: the biological samples were transferred into Eppendorf tubes and stored at −80° C.

The samples were generated from two sets of explants prepared from two independent experiments.

### Results

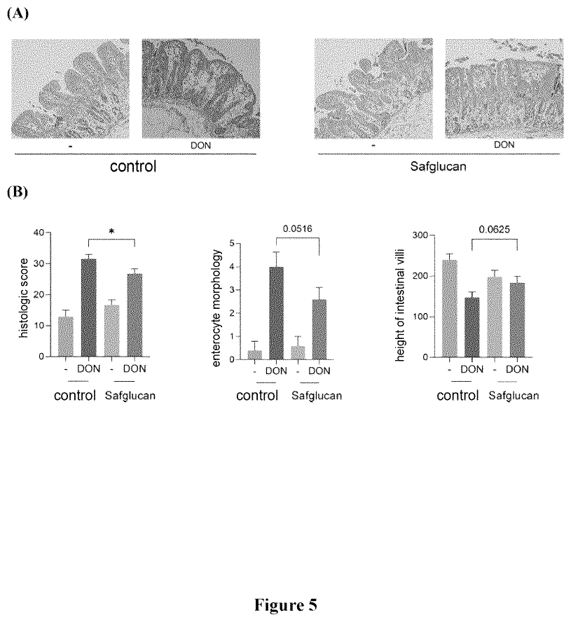

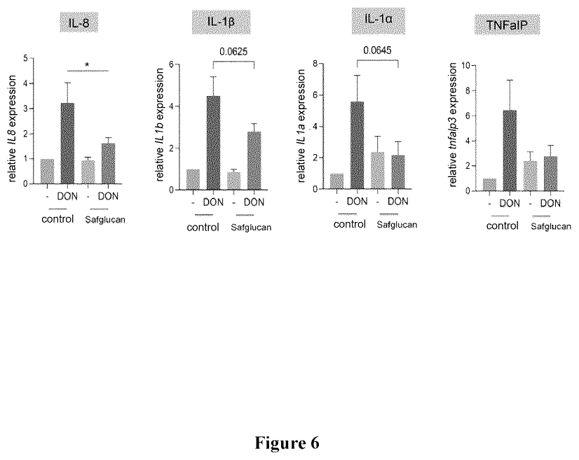

The results of the ex vivo studies are shown in FIGS. 5 and 6.

5 μm sections were prepared from the paraffin blocks containing the treated jejunum explants. They were then stained with hematoxylin-eosin (H&E) for histopathological analysis, which takes into account different types of lesions. The results obtained are shown in FIG. 5. These results show that DON increases the number of lesions on the tissue, and Safglucan® makes it possible to reduce the damage to the villi of explants co-exposed to DON. Enterocyte damage characterized by the observation of flattening of jejunal epithelial cells in explants co-exposed to DON is also reduced in the presence of Safglucan®. A significant decrease of approximately 35% in villus length was observed in jejunum explants treated with DON (10 mM). Treatment of explants with Safglucan® tends to reduce the impact of DON on villi length.

The RNA from the explants was then extracted and used to study the expression of different genes, particularly genes related to inflammation and cytokine production. The results obtained are shown in FIG. 6. These results show that exposure of explants to 10 μM DON leads to an increase in the production of gene transcripts linked to inflammation. The presence of Safglucan® significantly downmodulates the expression of transcripts for the il1b and il8 genes. A downward trend was also observed for il1a.

### Example 4: Evaluation of the Effects of Safglucan® In Vivo

The experiments described in this example were carried out by Regiane Santos at Schothorst Feed Research, whose address is: Meerkoetenweg, 26 8218 NA Lelystad, The Netherlands.

The in vivo studies were carried out on broiler chickens exposed to feed naturally contaminated with DON mycotoxin.

### Materials and Methods

The experiment lasted 28 days, with an initial startup phase of 0 to 13 days and a growth phase of 13 to 28 days. The experiment included 3 treatments according to a “randomized complete block design” experimental design, each having replicates, each replicate comprising 24 cages, and each cage 13 animals.

TABLE 3
Dietary treatment.

| | T1 | T2 | T3 | |
|---|---|---|---|---|
| DON | <0.5 | 3.5 ppm | 3.5 | ppm |
| Safglucan ® | 0 | 0 | 125 | g/ton |

On days 13 and 28, three animals per cage and per treatment (i.e., 18 animals per treatment) were randomly selected and euthanized. The liver and the jejunum were removed. The livers were observed macroscopically, and the pale color and the lesions, such as cysts or hemorrhagic areas, were counted and expressed as percentage relative to the livers not exhibiting lesions. Using the jejunum, the height of the villi and the depth of the crypts were measured on a minimum of 5 villi per animal, per treatment and per cage.

### Results

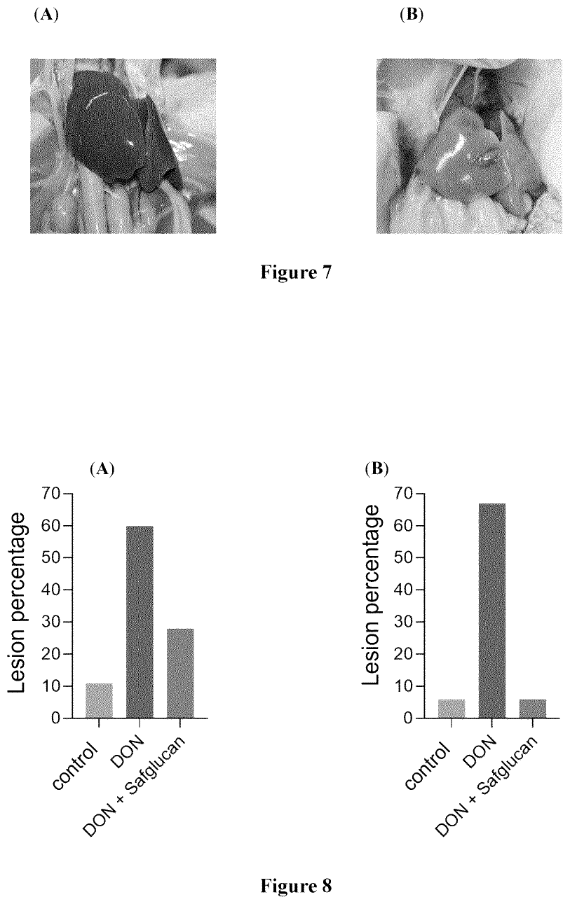

The results of the in vivo studies are shown in FIGS. 7 and 8.

FIG. 7 shows the lesions of a chicken liver treated with DON mycotoxin (left) compared to an untreated liver. The liver exposed to DON mycotoxin appears paler, slightly yellower; a hemorrhagic lesion is clearly visible.

FIG. 8 shows graphs indicating the percentage of liver lesions observed in chickens on days 13 and 28 depending on the presence or absence of DON mycotoxin and/or Safglucan® in the diet. It can be clearly seen that the presence of DON in feed induces a strong increase in lesions in the liver, and the presence of Safglucan® in the diet made it possible to prevent lesions induced by DON mycotoxin.

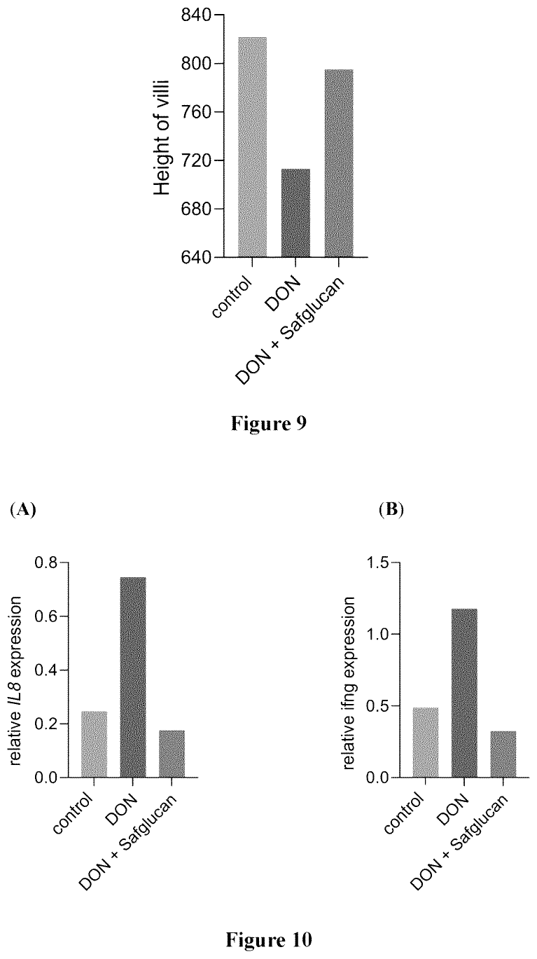

FIG. 9 is a graph showing the height of intestinal villi in the jejunum of chickens on day 13 depending on the presence or absence of DON mycotoxin and/or Safglucan® in the diet. It can be seen that the presence of DON in the feed induces a reduction in the size of the intestinal villi of the jejunum, and the presence of Safglucan® in the diet made it possible to prevent this reduction in the size of the villi.

FIG. 10 shows graphs indicating the expression of inflammatory genes in the jejunum of chickens depending on the presence or absence of DON mycotoxin and/or Safglucan® in the diet. It can be seen that the presence of DON in feed induces a strong increase in il8 and interferon gamma (ifnγ) mRNA, and the presence of Safglucan® in the diet made it possible to prevent this increase in expression of these two genes linked to inflammation.

1-13. (canceled)

14. A method for preventing mycotoxicosis caused by mycotoxin deoxynivalenol (DON) in a subject in need thereof, the method comprising a step of administering to said subject an extract of β-glucan-rich Saccharomyces cerevisiae yeast cell walls comprising β-glucans and mannans, wherein the β-glucans are present in the form of a mixture of β-1,3-glucans and β-1,6-glucans in a total amount greater than or equal to 50% by weight; and the mannans are present in a total amount of less than 5% by weight.

15. The method according to claim 14, wherein the b-glucans and the mannans are the only active ingredients in said extract of β-glucan-rich Saccharomyces cerevisiae yeast cell walls.

16. The method according to claim 14, wherein the mannans are present in an amount such that the β-glucans/mannans ratio is between 12 and 40 (wt/wt).

17. The method according to claim 14, wherein said extract has a dry matter content greater than or equal to 94% by weight.

18. The method according to claim 14, wherein said extract further comprises a protein content less than or equal to 10% by weight and a glycogen content less than or equal to 10% by weight.

19. The method according to claim 14, wherein said extract is Safglucan®, which comprises b-glucans and mannans, wherein the b-glucans are present in the form of a mixture of b-1,3-glucans and b-1,6-glucans in a total amount between 52 and 60% by weight; and the mannans are present in a total amount between 1 and 5% by weight, and wherein the Safglucan® further comprises a protein content less than or equal to 10% by weight and a glycogen content of less then or equal to 10% by weight.

20. The method according to claim 14, wherein said extract of β-glucan-rich Saccharomyces cerevisiae yeast cell walls is comprised in a pharmaceutical composition, said pharmaceutical composition further comprising at least one physiologically acceptable excipient.

21. The method according to claim 14, wherein said extract of β-glucan-rich Saccharomyces cerevisiae yeast cell walls is comprised in a composition, said composition further comprising at least one bioactive agent.

22. The method according to claim 21, wherein said bioactive agent is selected from the group consisting of mycotoxin binders, mycotoxin biotransformants, vitamins, minerals, trace elements, hepatoprotectants, immunoprotectants, and combinations thereof.

23. The method according to claim 14, wherein the subject is an animal.

24. The method according to claim 23, wherein said extract of β-glucan-rich Saccharomyces cerevisiae yeast cell walls is comprised in an animal feed.

25. A method for preventing immunotoxic effects of mycotoxin deoxynivalenol (DON) in a subject in need thereof, the method comprising a step of administering to said subject, an extract of β-glucan-rich Saccharomyces cerevisiae yeast cell walls comprising β-glucans and mannans, wherein the β-glucans are present in the form of a mixture of β-1,3-glucans and β-1,6-glucans in a total amount greater than or equal to 50% by weight; and the mannans are present in a total amount of less than 5% by weight.

26. The method according to claim 25, wherein the b-glucans and the mannans are the only active ingredients in said extract of β-glucan-rich Saccharomyces cerevisiae yeast cell walls.

27. The method according to claim 25, wherein the mannans are present in an amount such that the β-glucans/mannans ratio is between 12 and 40 (wt/wt).

28. The method according to claim 25, wherein said extract has a dry matter content greater than or equal to 94% by weight.

29. The method according to claim 25, wherein said extract further comprises a protein content less than or equal to 10% by weight and a glycogen content less than or equal to 10% by weight.

30. The method according to claim 25, wherein said extract is Safglucan®, which comprises b-glucans and mannans, wherein the b-glucans are present in the form of a mixture of b-1,3-glucans and b-1,6-glucans in a total amount between 52 and 60% by weight; and the mannans are present in a total amount between 1 and 5% by weight, and wherein the Safglucan® further comprises a protein content less than or equal to 10% by weight and a glycogen content of less then or equal to 10% by weight.

31. The method according to claim 25, wherein said extract of β-glucan-rich Saccharomyces cerevisiae yeast cell walls is comprised in a pharmaceutical composition, said pharmaceutical composition further comprising at least one physiologically acceptable excipient.

32. The method according to claim 25, wherein said extract of β-glucan-rich Saccharomyces cerevisiae yeast cell walls is comprised in a composition, said composition further comprising at least one bioactive agent.

33. The method according to claim 32, wherein said bioactive agent is selected from the group consisting of mycotoxin binders, mycotoxin biotransformants, vitamins, minerals, trace elements, hepatoprotectants, immunoprotectants, and combinations thereof.

34. The method according to claim 25, wherein the subject is an animal.

35. The method according to claim 34, wherein said extract of β-glucan-rich Saccharomyces cerevisiae yeast cell walls is comprised in an animal feed.

36. A method for preventing hepatic and/or intestinal lesions caused by mycotoxin deoxynivalenol (DON) in a subject in need thereof, the method comprising a step of administering to said subject, an extract of β-glucan-rich Saccharomyces cerevisiae yeast cell walls comprising β-glucans and mannans, wherein the β-glucans are present in the form of a mixture of β-1,3-glucans and β-1,6-glucans in a total amount greater than or equal to 50% by weight; and the mannans are present in a total amount of less than 5% by weight.

37. The method according to claim 36, wherein the b-glucans and the mannans are the only active ingredients in said extract of β-glucan-rich Saccharomyces cerevisiae yeast cell walls.

38. The method according to claim 36, wherein the mannans are present in a β-glucans/mannans ratio that is between 12 and 40 (wt/wt).

39. The method according to claim 36, wherein said extract has a dry matter content greater than or equal to 94% by weight.

40. The method according to claim 36, wherein said extract further comprises a protein content less than or equal to 10% by weight and a glycogen content less than or equal to 10% by weight.

41. The method according to claim 36, wherein said extract is Safglucan®, which comprises b-glucans and mannans, wherein the b-glucans are present in the form of a mixture of b-1,3-glucans and b-1,6-glucans in a total amount between 52 and 60% by weight; and the mannans are present in a total amount between 1 and 5% by weight, and wherein the Safglucan® further comprises a protein content less than or equal to 10% by weight and a content of less then or equal to 10% by weight.

42. The method according to claim 36, wherein said extract of β-glucan-rich Saccharomyces cerevisiae yeast cell walls is comprised in a pharmaceutical composition, said pharmaceutical composition further comprising at least one physiologically acceptable excipient.

43. The method according to claim 36, wherein said extract of β-glucan-rich Saccharomyces cerevisiae yeast cell walls is comprised in a composition, said composition further comprising at least one bioactive agent.

44. The method according to claim 43, wherein said bioactive agent is selected from the group consisting of mycotoxin binders, mycotoxin biotransformants, vitamins, minerals, trace elements, hepatoprotectants, immunoprotectants, and combinations thereof.

45. The method according to claim 36, wherein the subject is an animal.

46. The method according to claim 45, wherein said extract of β-glucan-rich Saccharomyces cerevisiae yeast cell walls is comprised in an animal feed.
